(19) United States
(12) Patent Application Publication
(10) Pub. No.: US 2019/0008149 A1
(43) Pub. Date: Jan. 10, 2019

(54) HERBICIDAL COMPOSITIONS CONTAINING 4-AMINO-3-CHLORO-6-(4-CHLORO-2-FLUORO-3-METHOXYPHENYL) PYRIDINE-2-CARBOXYLIC ACID OR A DERIVATIVE THEREOF AND A CELL MEMBRANE DISRUPTOR HERBICIDE OR A DERIVATIVE THEREOF

(71) Applicant: Dow AgroSciences LLC, Indianapolis, IN (US)

(72) Inventors: Christopher J. Voglewede, Calgary (CA); Richard K. Mann, Indianapolis, IN (US); Luiz Henrique Zobiole, Toledo (BR); Augusto Kalsing, Mogi Mirim (BR); Gizelly Santos, Campo Grande (BR)

(73) Assignee: Dow AgroSciences LLC, Indianapolis, IN (US)

(21) Appl. No.: 16/067,262

(22) PCT Filed: Sep. 8, 2016
(86) PCT No.: PCT/US2016/050699

§ 371 Date: Jun. 29, 2018

Related U.S. Application Data
(60) Provisional application No. 62/272,205, filed on Dec. 29, 2015.

(51) Int. Cl. A01N 43/40; C07D 213/79; A01N 43/54; A01N 25/32

(52) U.S. Cl. CPC A01N 43/40; A01N 25/32; A01N 43/54; C07D 213/79

(57) ABSTRACT

Herbicidal compositions and methods using a combination of (a) a compound of formula (I):

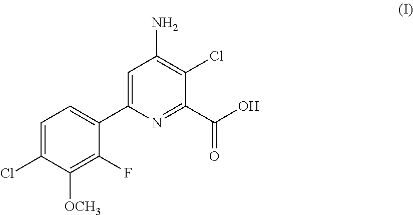 [SMILES: COc1c(Cl)ccc(-c2cc(N)c(Cl)c(C(=O)O)n2)c1F]

or an agriculturally acceptable salt or ester thereof and (b) a cell membrane disruptor herbicide or an agriculturally acceptable salt or ester thereof provide control of undesirable vegetation in soybean, cotton, sunflower, winter/spring oilseed rape, winter/spring canola, vegetables, ornamentals, rice, wheat, teff, triticale, barley, oats, rye, sorghum, corn/maize, sunflower, row crops, pastures, grasslands, rangelands, fallowland, sugarcane, turf, tree and vine orchards, aquatics, forestry, industrial vegetation management (IVM) and rights-of-way; for the burndown and control of weeds in fallow fields and fallow beds between the planting of crops, such as soybean, corn, cotton, sunflower, cereals, rice, winter/spring oilseed rape, winter/spring canola, corn/maize and sorghum; and for the control of weeds just prior to or after planting soybeans.

### BACKGROUND

The protection of crops from weeds and other vegetation which inhibit crop growth is a constantly recurring problem in agriculture. To help combat this problem, researchers in the field of synthetic chemistry have produced an extensive variety of chemicals and chemical formulations effective in the control of such unwanted growth. Chemical herbicides of many types have been disclosed in the literature and a large number are in commercial use. However, there remains a need for compositions and methods that are effective in controlling undesirable vegetation.

### SUMMARY

Provided herein are herbicidal compositions containing a herbicidally effective amount of (a) a compound of the formula (I)

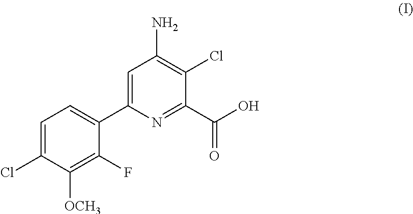 [SMILES: COc1c(Cl)ccc(-c2cc(N)c(Cl)c(C(=O)O)n2)c1F]

or an agriculturally acceptable salt or ester of thereof and (b) a cell membrane disruptor herbicide or an agriculturally acceptable salt or ester thereof.

In some embodiments, (b) is a photosystem I (PS I) inhibitor. In another embodiment, the PS I inhibitor includes diquat and paraquat, agriculturally acceptable salts thereof, and combinations thereof.

In some embodiments, (b) is a protoporphyrinogen oxidase (PPO) inhibitor, wherein (b) is not carfentrazone or a salt or ester thereof. In some embodiments, the PPO inhibitor herbicides include acifluorfen, bifenox, chlomethoxyfen, fluoroglycofen, fomesafen, halosafen, lactofen, oxyfluorfen, fluazolate, pyraflufen, cinidon, flumioxazin, flumiclorac, fluthiacet, thidiazimin, oxadiazon, oxadiargyl, azafenidin, sulfentrazone, pentoxazone, trifludimoxazin, benzfendizone, butafenacil, saflufenacil, pyraclonil, profluazol, flufenpyr, and agriculturally acceptable salts and esters thereof, and combinations thereof.

The compositions may also contain one or more agriculturally acceptable safeners, adjuvants and/or carriers. In some embodiments, the activity of the mixtures is greater than the sum of the activity of the individual active ingredients.

The two-way mixture can be prepared as a tank mix or premix. For example, a tank mix or premix containing the compound of formula (I) or a salt or ester thereof and a cell membrane disruptor herbicide or an agriculturally acceptable salt or ester thereof form the two-component tank mix. In this embodiment, the tank mix, premix or concentrate of the compound of formula (I) and a disruptor herbicide or an agriculturally acceptable salt or ester thereof can be packaged and sold separately, can be copackaged together in separate containers or can be formulated together in one package.

In other embodiments, a concentrate containing the compound of formula (I) or a salt or ester thereof can be mixed in a tank or container with a cell membrane disruptor herbicide or an agriculturally acceptable salt or ester thereof to form the tank mix. In this embodiment, the concentrate of the compound of formula (I) and the cell membrane disruptor herbicide or an agriculturally acceptable salt or ester thereof can be packaged and sold separately, or can be copackaged together in separate containers, or can be formulated together in one package.

The ratio of the compound of formula (I) to a cell membrane disruptor herbicide or a salt or ester thereof in the premix or tank mix is from about 1:4800 to 100:1; from about 1:1150 to 30:1, and from about 1:400 to about 4:1.

The ratio of the compound of formula (I) to a PS I inhibitor herbicide or a salt thereof in the premix or tank mix is from about 1:4480 to 2.5:1; from about 1:900 to 0.5:1, and from about 1:180 to about 0.20:1. The ratio of the compound of formula (I) to a PPO inhibitor herbicide or a salt or ester thereof in the premix or tank mix is from about 1:4800 to 100:1, from about 1:1150 to 30:1, and from about 1:400 to 4:1.

Also provided are methods of controlling undesirable vegetation comprising applying (a) a compound of formula (I) or an agriculturally acceptable ester or salt thereof and (b) a cell membrane disruptor herbicide or an agriculturally acceptable salt or ester thereof.

### DETAILED DESCRIPTION

### I. Definitions

As used herein, control of or controlling undesirable vegetation means killing or preventing the growth of the vegetation, or causing some other adverse modifying effect to the vegetation e.g., necrosis, chlorosis, stunting, deviations from natural growth or development, regulation, desiccation, retardation, and the like.

As used herein, herbicide and herbicidal active ingredient mean a compound that controls undesirable vegetation when applied in an appropriate amount.

As used herein, a herbicidally effective or vegetation controlling amount is an amount of herbicidal active ingredient the application of which controls the relevant undesirable vegetation.

As used herein, applying a herbicide or herbicidal composition means delivering it directly to the targeted vegetation or to the locus thereof or to the area where control of undesired vegetation is desired. Methods of application include, but are not limited to pre-emergence, post-emergence, foliar and soil applications. Described herein are methods of controlling undesirable vegetation by applying certain herbicide combinations or compositions.

As used herein, plants and vegetation include, but are not limited to, dormant seeds, germinant seeds, emerging seedlings, plants emerging from vegetative propagules, immature vegetation, mature vegetation, reproductive vegetation and established vegetation.

As used herein, cell membrane disruptor herbicides show burning or yellowing of plant tissues.

As used herein, immature vegetation refers to small vegetative plants prior to reproductive stage, and mature vegetation refers to vegetative plants during and after reproductive stage.

### II. Mixtures

Compositions containing the compound of formula (I) or a salt or ester thereof and a disruptor herbicide or an agriculturally acceptable salt or ester thereof are described herein. The composition can be in the form of a tank mix, premix, or co-pack containing the compound of formula (I) or a salt or ester thereof and a disruptor herbicide or an agriculturally acceptable salt or ester thereof. The tank mix can be prepared by mixing a premix, co-pack or concentrate containing the compound of formula (I) and a disruptor herbicide or an agriculturally acceptable salt or ester thereof. In some embodiments, (b) is a photosystem I (PS I) inhibitor. In another embodiment, the PS I inhibitor includes diquat and paraquat, agriculturally acceptable salts thereof, and combinations thereof. In some embodiments, (b) is a protoporphyrinogen oxidase (PPO) inhibitor, wherein (b) is not carfentrazone or a salt or ester thereof. In some embodiments, the PPO inhibitor herbicides include acifluorfen, bifenox, chlomethoxyfen, fluoroglycofen, fomesafen, halosafen, lactofen, oxyfluorfen, fluazolate, pyraflufen, cinidon, flumioxazin, flumiclorac, fluthiacet, thidiazimin, oxadiazon, oxadiargyl, azafenidin, sulfentrazone, pentoxazone, trifludimoxazin, benzfendizone, butafenacil, saflufenacil, pyraclonil, profluazol, flufenpyr, and agriculturally acceptable salts and esters thereof, and combinations thereof.

Agriculturally acceptable salts and esters refer to salts and esters that exhibit herbicidal activity, or that are or can be converted in plants, water, or soil to the referenced herbicide. Exemplary agriculturally acceptable esters are those that are or can by hydrolyzed, oxidized, metabolized, or otherwise converted, e.g., in plants, water, or soil, to the corresponding carboxylic acid which, depending on the pH, may be in the dissociated or undissociated form.

Exemplary salts include those derived from alkali or alkaline earth metals and those derived from ammonia and amines Exemplary cations include sodium, potassium, magnesium, and ammonium cations of the formula:

R¹R²R³R⁴N⁺

wherein R¹, R², R³ and R⁴ each, independently represents hydrogen or C₁-C₁₂ alkyl, C₃-C₁₂ alkenyl or C₃-C₁₂ alkynyl, each of which is optionally substituted by one or more hydroxy, C₁-C₄ alkoxy, C₁-C₄ alkylthio or phenyl groups, provided that R¹, R², R³ and R⁴ are sterically compatible. Additionally, any two of R¹, R², R³ and R⁴ together may represent an aliphatic difunctional moiety containing one to twelve carbon atoms and up to two oxygen or sulfur atoms. Salts can be prepared by treatment with a metal hydroxide, such as sodium hydroxide, with an amine, such as ammonia, trimethylamine, diethanolamine, 2-methylthiopropylamine, bisallylamine, 2-butoxyethylamine, morpholine, cyclododecylamine, or benzylamine or with a tetraalkylammonium hydroxide, such as tetramethylammonium hydroxide or choline hydroxide.

Exemplary esters include those derived from C₁-C₁₂ alkyl, C₃-C₁₂ alkenyl, C₃-C₁₂ alkynyl or C₇-C₁₀ aryl-substituted alkyl alcohols, such as methyl alcohol, isopropyl alcohol, 1-butanol, 2-ethylhexanol, butoxyethanol, methoxypropanol, allyl alcohol, propargyl alcohol, cyclohexanol or unsubstituted or substituted benzyl alcohols. Benzyl alcohols may be substituted with from 1-3 substituents independently selected from halogen, C₁-C₄ alkyl or C₁-C₄ alkoxy. Esters can be prepared by coupling of the acids with the alcohol using any number of suitable activating agents such as those used for peptide couplings such as dicyclohexylcarbodiimide (DCC) or carbonyl diimidazole (CDI); by reacting the acids with alkylating agents such as alkylhalides or alkylsulfonates in the presence of a base such as triethylamine or lithium carbonate; by reacting the corresponding acid chloride of an acid with an appropriate alcohol; by reacting the corresponding acid with an appropriate alcohol in the presence of an acid catalyst or by transesterification.

Weight ratios of mixtures are calculated using the acid equivalent weight(s) of any compounds in the mixture that are salts or esters. Weight ratios are expressed in units of weight to weight (grams (g) to g, grams acid equivalent per hectare (g ae/ha) to g ae/ha, grams acid equivalent per hectare (g ae/ha) to grams active ingredient per hectare (g ai/ha), or grams active ingredient per hectare (g ai/ha) to g ai/ha).

In some embodiments, the combination of compound (I) or agriculturally acceptable salt or ester thereof and a disruptor herbicide or an agriculturally acceptable salt or ester thereof may be more effective than the activity of the individual herbicidal active ingredients.

A. Halauxifen

The compound of formula (I) has the following structure:

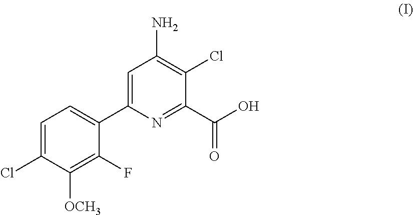 [SMILES: COc1c(Cl)ccc(-c2cc(N)c(Cl)c(C(=O)O)n2)c1F]

The compound of formula (I) can be identified by the name 4-amino-3-chloro-6-(4-chloro-2-fluoro-3-methoxyphenyl)pyridine-2-carboxylic acid and has been described in U.S. Pat. No. 7,314,849 (B2), which is incorporated herein by reference in its entirety. It is also referred to as halauxifen. The methyl ester is known as halauxifen-methyl and is sold commercially as ARYLEX®.

In certain embodiments of the compositions and methods described herein, the compound of formula (I), i.e., the carboxylic acid, is employed. In certain embodiments, a carboxylate salt of the compound of formula (I) is employed. In certain embodiments, an arylalkyl or alkyl ester is employed. In certain embodiments, a benzyl, substituted benzyl, or C₁-C₄ alkyl, e.g., methyl or n-butyl ester is employed. In certain embodiments, the methyl ester, benzyl ester, or potassium salt is employed.

Exemplary uses of the compound of the formula (I) include controlling undesirable vegetation, including e.g., grass, broadleaf and sedge weeds, in cereal and multiple perennial and non-cropping situations.

B. Cell Membrane Disruptor Herbicides

As used herein, cell membrane disruptor herbicides show burning, yellowing and/or necrosis of plant tissues. In some embodiments, (b) is a photosystem I (PS I) inhibitor. In another embodiment, the PS I inhibitor includes diquat and paraquat, agriculturally acceptable salts thereof, and combinations thereof. In some embodiments, (b) is a protoporphyrinogen oxidase (PPO) inhibitor, wherein (b) is not carfentrazone or a salt or ester thereof. In some embodiments, the PPO inhibitor herbicides include acifluorfen, bifenox, chlomethoxyfen, fluoroglycofen, fomesafen, halosafen, lactofen, oxyfluorfen, fluazolate, pyraflufen, cinidon, flumioxazin, flumiclorac, fluthiacet, thidiazimin, oxadiazon, oxadiargyl, azafenidin, sulfentrazone, pentoxazone, trifludimoxazin, benzfendizone, butafenacil, saflufenacil, pyraclonil, profluazol, flufenpyr, and agriculturally acceptable salts and esters thereof, and combinations thereof.

Paraquat, 1,1′-dimethyl-4,4′-bipyridinediium or 1,1′-dimethyl-4,4′-bipyridylium, is a non-selective contact herbicide, with some translocation in the xylem. Paraquat, which has the following structure:

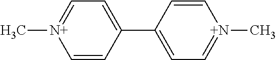

is used for broad-spectrum control of broadleaf weeds and grasses in a variety of crops; for general weed control on non-crop land; as a defoliant for cotton and hops; for destruction of potato haulms; as a desiccant for pineapples, sugar cane, soybeans and sunflowers; for strawberry runner control; and in pasture renovation. Paraquat dichoride is commercialized in an aqueous solution.

Saflufenacil, 2-chloro-5-[3,6-dihydro-3-methyl-2,6-dioxo-4-(trifluoromethyl)-1(2H)-pyrimidinyl]-4-fluoro-N-[[methyl(1-methylethyl)amino]sulfonyl]benzamide, is a foliar contact and residual broadleaf weed herbicide. Saflufenacil, which has the following structure:

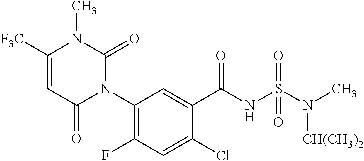 [SMILES: CC(C)N(C)S(=O)(=O)NC(=O)c1cc(-n2c(=O)cc(C(F)(F)F)n(C)c2=O)c(F)cc1Cl]

is used for foliar and residual control of broadleaf weeds, including glyphosate- and acetolactate synthase (ALS)-resistant biotypes. It is absorbed by the foliage and roots, with translocation in the apoplast and limited movement in the phloem. Saflufenacil is applied pre-emergence in corn and sorghum, at 50-125 g/ha; pre-plant for rapid foliar burndown in soybeans, cereals, cotton, legumes, and post-directed in tree fruit and nuts, at 18-25 g/ha. Saflufenacil's mode of action is to inhibit protoporphyrinogen oxidase (PPO).

Saflufenacil is sold commercially under a number of names including Heat, Sharpen, Treevix, Valeos and Eragon (BASF Corporation).

Other examples of PPO inhibitors include those described in U.S. Pat. No. 8,754,008 (B2), such as trifludimoxazin, which has the following structure:

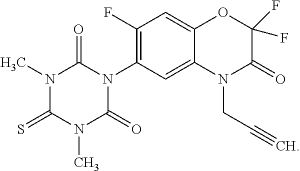 [SMILES: C#CCN1C(=O)C(F)(F)Oc2cc(F)c(-n3c(=O)n(C)c(=S)n(C)c3=O)cc21]

### III. Tank Mixes, PreMixes and Co-Packs

Tank mixes, premixes and co-packs containing the compound of formula (I) or a salt or ester thereof and a cell membrane disruptor herbicide or an agriculturally acceptable salt or ester thereof are described herein. In some embodiments, the tank mix is prepared by mixing a premix of the compound of formula (I) or a salt or ester thereof with a concentrate containing a disruptor herbicide or an agriculturally acceptable salt or ester thereof. In some embodiments, (b) is a photosystem I (PS I) inhibitor. In another embodiment, the PS I inhibitor includes diquat and paraquat, agriculturally acceptable salts thereof, and combinations thereof. In some embodiments, (b) is a protoporphyrinogen oxidase (PPO) inhibitor, wherein (b) is not carfentrazone or a salt or ester thereof. In some embodiments, the PPO inhibitor herbicides include acifluorfen, bifenox, chlomethoxyfen, fluoroglycofen, fomesafen, halosafen, lactofen, oxyfluorfen, fluazolate, pyraflufen, cinidon, flumioxazin, flumiclorac, fluthiacet, thidiazimin, oxadiazon, oxadiargyl, azafenidin, sulfentrazone, pentoxazone, trifludimoxazin, benzfendizone, butafenacil, saflufenacil, pyraclonil, profluazol, flufenpyr, and agriculturally acceptable salts and esters thereof, and combinations thereof. In other embodiments, the premix or co-pack contains both active ingredients and is diluted prior to use/application. The premix or suitable premixes can contain one or more suitable adjuvants and/or carriers.

In some embodiments, compositions provided herein contain at least one agriculturally acceptable adjuvant or carrier. Suitable adjuvants or carriers should not be phytotoxic to valuable crops, particularly at the concentrations employed in applying the compositions for selective weed control in the presence of crops, and should not react chemically with herbicidal components or other composition ingredients. Such mixtures can be designed for application directly to weeds or their locus or can be concentrates or formulations that are normally diluted with additional carriers and adjuvants before application. They can be solids, such as, for example, dusts, granules, water-dispersible granules, or wettable powders, or liquids, such as, for example, emulsifiable concentrates, solutions, emulsions or suspensions. They can also be provided as a premix or tank-mixed.

Suitable agricultural adjuvants and carriers include, but are not limited to, crop oil concentrate; nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂-C₁₆) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG(400) dioleate-99.

Liquid carriers that can be employed include water and organic solvents. The organic solvents include, but are not limited to, petroleum fractions or hydrocarbons such as mineral oil, aromatic solvents, paraffinic oils, and the like; vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like; esters of the above vegetable oils; esters of monoalcohols or dihydric, trihydric, or other lower polyalcohols (4-6 hydroxy containing), such as 2-ethyl hexyl stearate, n-butyl oleate, isopropyl myristate, propylene glycol dioleate, di-octyl succinate, di-butyl adipate, di-octyl phthalate and the like; esters of mono, di and polycarboxylic acids and the like. Specific organic solvents include, but are not limited to toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers and the like. In certain embodiments, water is the carrier for the dilution of concentrates.

Suitable solid carriers include but are not limited to talc, pyrophyllite clay, silica, attapulgus clay, kaolin clay, kieselguhr, chalk, diatomaceous earth, lime, calcium carbonate, bentonite clay, Fuller's earth, cottonseed hulls, wheat flour, soybean flour, pumice, wood flour, walnut shell flour, lignin, cellulose, and the like.

In some embodiments, the compositions described herein further comprise one or more surface-active agents. In some embodiments, such surface-active agents are employed in both solid and liquid compositions, and in certain embodiments those designed to be diluted with carrier before application. The surface-active agents can be anionic, cationic or nonionic in character and can be employed as emulsifying agents, wetting agents, suspending agents, or for other purposes. Surfactants which may also be used in the present formulations are described, inter alia, in McCutcheon's Detergents and Emulsifiers Annual, MC Publishing Corporation: Ridgewood, N J, 1998 and in Encyclopedia of Surfactants, Vol. I-III, Chemical Publishing Company: New York, 1980-81. Surface-active agents include, but are not limited to salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C₁₈ ethoxylate; alcohol-alkylene oxide addition products, such as tridecyl alcohol-C₁₆ ethoxylate; soaps, such as sodium stearate; alkyl-naphthalene-sulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable or seed oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, and in certain embodiments, methyl esters.

In some embodiments, these materials, such as vegetable or seed oils and their esters, can be used interchangeably as an agricultural adjuvant, as a liquid carrier or as a surface active agent.

Other exemplary additives for use in the compositions provided herein include but are not limited to compatibilizing agents, antifoam agents, sequestering agents, neutralizing agents and buffers, corrosion inhibitors, dyes, odorants, spreading agents, penetration aids, sticking agents, dispersing agents, thickening agents, freezing point depressants, antimicrobial agents, and the like. The compositions may also contain other compatible components, for example, other herbicides, plant growth regulants, fungicides, insecticides, and the like and can be formulated with liquid fertilizers or solid, particulate fertilizer carriers such as ammonium nitrate, urea and the like.

The tank mixes described herein can further contain one or more additional herbicides. The herbicides can be incorporated into the one or more of the premixes, tank mixes or concentrates or can be added at the time the tank mix is prepared by formulation or prior to application. When used in conjunction with other herbicides, the composition can be formulated with the other herbicide or herbicides, tank-mixed with the other herbicide or herbicides or applied sequentially with the other herbicide or herbicides. Some of the herbicides that can be employed in conjunction with the compositions and methods described herein include, but are not limited to: 4-CPA, 4-CPB, 4-CPP, 2,4-D, 2,4-D choline salt, 2,4-D esters and amines, 2,4-DB, 3,4-DA, 3,4-DB, 2,4-DEB, 2,4-DEP, 3,4-DP, 2,3,6-TBA, 2,4,5-T, 2,4,5-TB, acetochlor, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azimsulfuron, aziprotryne, barban, BCPC, beflubutamid, benazolin, bencarbazone, benfluralin, benfuresate, bensulfuron-methyl, bensulide, benthiocarb, bentazon-sodium, benzadox, benzipram, benzobicyclon, benzofenap, benzofluor, benzoylprop, benzthiazuron, bicyclopyrone, bilanafos, bispyribac-sodium, borax, bromacil, bromobonil, bromobutide, bromofenoxim, bromoxynil, brompyrazon, butachlor, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole, chlorprocarb, CDEA, CEPC, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlornitrofen, chloropon, chlorotoluron, chloroxuron, chloroxynil, chlorpropham, chlorsulfuron, chlorthal, chlorthiamid, cinmethylin, cinosulfuron, cisanilide, clacyfos, clethodim, cliodinate, clodinafop-propargyl, clofop, clomazone, clomeprop, cloprop, cloproxydim, clopyralid, CMA, copper sulfate, CPMF, CPPC, credazine, cresol, cumyluron, cyanatryn, cyanazine, cycloate, cyclopyrimorate, cyclosulfamuron, cycloxydim, cycluron, cyhalofop-butyl, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop-methyl, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal, epronaz, EPTC, erbon, esprocarb, ethalfluralin, ethbenzamide, ethametsulfuron, ethidimuron, ethiolate, ethobenzanid, ethofumesate, ethoxyfen, ethoxysulfuron, etinofen, etnipromid, etobenzanid, EXD, fenasulam, fenoprop, fenoxaprop, fenoxaprop-P-ethyl, fenoxaprop-P-ethyl+isoxadifen-ethyl, fenoxasulfone, fenquinotrione, fenteracol, fenthiaprop, fentrazamide, fenuron, ferrous sulfate, flamprop, flamprop-M, flazasulfuron, florpyrauxifen, fluazifop, fluazifop-P-butyl, flucarbazone, flucetosulfuron, fluchloralin, flufenacet, flufenican, flumezin, flumipropyn, fluometuron, fluorodifen, fluoromidine, fluoronitrofen, fluothiuron, flupoxam, flupropacil, flupropanate, flupyrsulfuron, fluridone, flurochloridone, fluroxypyr, flurtamone, foramsulfuron, fosamine, fumiclorac, furyloxyfen, glufosinate, glyphosate, halosulfuron-methyl, haloxydine, haloxyfop-methyl, haloxyfop-P-methyl, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, iodosulfuron-ethyl-sodium, iofensulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lancotrione, lenacil, linuron, MAA, MAMA, MCPA esters and amines, MCPA-thioethyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metoxuron, metribuzin, metsulfuron, metsulfuron-methyl, molinate, monalide, monisouron, monochloroacetic acid, monolinuron, monuron, morfamquat, MSMA, naproanilide, napropamide, napropamide-M, naptalam, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxapyrazon, oxasulfuron, oxaziclomefone, paraflufen-ethyl, parafluron, paraquat, pebulate, pelargonic acid, pendimethalin, pentachlorophenol, pentanochlor, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron-methyl, procyazine, prodiamine, profluazol, profluralin, profoxydim, proglinazine, prohexadione-calcium, prometon, prometryn, pronamide, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyrasulfotole, pyrazogyl, pyrazolynate, pyrazosulfuron-ethyl, pyrazoxyfen, pyribenzoxim, pyributicarb, pyriclor, pyridafol, pyridate, pyriftalid, pyriminobac, pyrimisulfan, pyrithiobac-sodium, pyroxasulfone, quinclorac, quinmerac, quinoclamine, quinonamid, quizalofop, quizalofop-P-ethyl, rhodethanil, rimsulfuron, S-metolachlor, sebuthylazine, secbumeton, sethoxydim, siduron, simazine, simeton, simetryn, SMA, sodium arsenite, sodium azide, sodium chlorate, sulcotrione, sulfallate, sulfometuron, sulfosate, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazuron, thiencarbazone-methyl, thifensulfuron, thifensulfuron-methyl, thiobencarb, tiafenacil, tiocarbazil, tioclorim, tolypyralate, topramezone, tralkoxydim, triafamone, tri-allate, triasulfuron, triaziflam, tribenuron, tribenuron-methyl, tricamba, triclopyr choline salt, triclopyr esters and salts, tridiphane, trietazine, trifloxysulfuron, trifluralin, triflusulfuron, trifop, trifopsime, trihydroxytriazine, trimeturon, tripropindan, tritac, tritosulfuron, vernolate, xylachlor and salts, choline salts, esters, optically active isomers and mixtures thereof.

In some embodiments, the compositions described herein are employed in combination with one or more herbicide safeners, such as AD-67 (MON 4660), benoxacor, benthiocarb, brassinolide, cloquintocet (mexyl), cyometrinil, cyprosulfamide, daimuron, dichlormid, dicyclonon, dimepiperate, disulfoton, fenchlorazole-ethyl, fenclorim, flurazole, fluxofenim, furilazole, harpin proteins, isoxadifen-ethyl, jiecaowan, jiecaoxi, mefenpyr-diethyl, mephenate, naphthalic anhydride (NA), oxabetrinil, R29148, and N-phenyl-sulfonylbenzoic acid amides, to enhance their selectivity. In some embodiments, the safeners are employed in soybean, cotton, sunflower, rice, canola, oilseed rapeseed, cereas (including but not limited to wheat, barley, oats and rye), sorghum, corn or maize settings.

In some embodiments, the compositions and methods described herein can be used in combination with one or more seed treatments known to be employed in the safening of rice and compounds of formula (I), including naphthalic anhydride and CAS registry number 129531-12-0 (N-(2-methoxybenzoyl)-4-Rmethylaminocarbonyllaminolbenzenesulfonamide), which has the following structure,

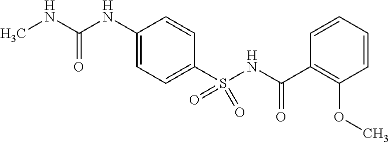

and CAS registry number 98967-94-3 (methyl 3-((5,7-dimethyl-[1,2,4]triazolo[1,5-a]pyrimidine)-2-sulfonamido)thiophene-2-carboxylate), which has the following structure,

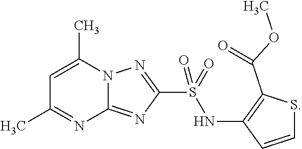 [SMILES: COC(=O)c1sccc1NS(=O)(=O)c1nc2nc(C)cc(C)n2n1]

In some embodiments, the safener is cloquintocet or an ester or salt thereof. In certain embodiments, cloquintocet is utilized to antagonize harmful effects of the compositions on rice and cereals. In some embodiments, the safener is cloquintocet (mexyl).

In some embodiments the methods provided herein are used to control undesirable vegetation in crops that are tolerant to glufosinate, dicamba, phenoxy auxins, pyridyloxy auxins, aryloxyphenoxypropionates, acetyl CoA carboxylase (ACCase) inhibitors, imidazolinones, acetolactate synthase (ALS) inhibitors, 4-hydroxyphenyl-pyruvate dioxygenase (HPPD) inhibitors, protoporphyrinogen oxidase (PPO) inhibitors, triazines, or bromoxynil. Such herbicide tolerant crops may possesses multiple or stacked traits conferring tolerance to multiple herbicides or multiple modes of action.

In some embodiments the methods provided herein are used to control undesirable vegetation that is a herbicide resistant or tolerant weed. Such herbicide resistant or tolerant weed may have a biotype with resistance or tolerance to single or multiple herbicides, single or multiple chemical classes, or single or multiple herbicide modes of action. For example, the herbicide resistant or tolerant weed may have a biotype resistant or tolerant to acetolactate synthase (ALS) inhibitors, photosystem II inhibitors, acetyl CoA carboxylase (ACCase) inhibitors, synthetic auxins, phenoxy auxins, pyridyloxy auxins, photosystem I inhibitors, 5-enolpyruvylshikimate-3-phosphate (EPSP) synthase inhibitors, microtubule assembly inhibitors, lipid synthesis inhibitors, protoporphyrinogen oxidase (PPO) inhibitors, carotenoid biosynthesis inhibitors, very long chain fatty acid (VLCFA) inhibitors, phytoene desaturase (PDS) inhibitors, glutamine synthetase inhibitors, 4-hydroxyphenyl-pyruvate-dioxygenase (HPPD) inhibitors, mitosis inhibitors, cellulose biosynthesis inhibitors, herbicides with multiple modes of action, quinclorac, arylaminopropionic acids, difenzoquat, endothall, or organoarsenicals.

In some embodiments, the concentration of the active ingredients in the compositions described herein is from about 0.0005 to 98 percent by weight. In some embodiments, the concentration is from about 0.0006 to 90 percent by weight. In compositions designed to be employed as concentrates, the active ingredients, in certain embodiments, are present in a concentration from about 0.1 to 98 weight percent, and in certain embodiments about 0.5 to 90 weight percent. Such compositions are, in certain embodiments, diluted with an inert carrier, such as water, before application. The diluted compositions usually applied to weeds or the locus of weeds contain, in certain embodiments, about 0.0003 to 10.0 weight percent active ingredient and in certain embodiments contain about 0.0008 to 5.0 weight percent.

### IV. Methods of Use

Herbicidal activity (control of undesirable vegetation in a crop or non-crop situation) is exhibited by the herbicidal compositions when they are applied directly to the plant or to the locus of the plant at any stage of growth, or to the area where control of vegetation is desired. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted to promote non-selective or selective herbicidal action. In some embodiments, the compositions described herein are applied as a post-emergence application to the crop and undesirable vegetation, pre-emergence application to the crop and post-emergence to the undesirable vegetation, and as multiple post-emergence applications to relatively immature undesirable vegetation to achieve the maximum control of weeds.

In some embodiments, the compositions and methods provided herein are utilized to control weeds in crops, including but not limited to soybean, cotton, sunflower, winter/spring oilseed rape, winter/spring canola, vegetables, ornamentals, rice, wheat, teff, triticale, barley, oats, rye, sorghum, corn/maize, sunflower, row crops, pastures, grasslands, rangelands, fallowland, sugarcane, turf, tree and vine orchards, aquatics, forestry, industrial vegetation management (IVM) and rights-of-way. In some embodiments, the compositions are used in the burndown and control of weeds in fallow fields and fallow beds between the planting of crops, such as soybean, corn, cotton, sunflower, cereals, corn/maize and sorghum. In other embodiments, the compositions are used to control weeds just prior to or after planting soybeans.

The compositions and methods provided herein are utilized to control undesirable vegetation. Undesirable vegetation includes, but is not limited to, undesirable vegetation that occurs in soybean, cotton, oilseed rape, peanuts, canola, vegetables, ornamentals, rice, wheat, teff, triticale, barley, oats, rye, sorghum, corn/maize, sunflower, row crops, pastures, grasslands, rangelands, fallowland, sugarcane, turf, tree and vine orchards, forestry, IVM and rights-of-way.

In some embodiments, the methods provided herein are utilized to control undesirable vegetation in fallow fields and fallow beds between crops, before planting soybean, cotton, corn/maize, sunflower, oilseed rape, canola, drilled crops and cereal crops, and after planting tolerant crops such as soybeans and other row crops. In certain embodiments, the undesirable vegetation is Alopecurus myosuroides Huds. (blackgrass, ALOMY), Apera spica-venti (L.) Beauv. (windgrass, APESV), Avena fatua L. (wild oat, AVEFA), Bromus tectorum L. (downy brome, BROTE), Lolium multiflorum Lam (Italian ryegrass, LOLMU), Lolium rigidum (rigid ryegrass, LOLRI), Lolium multiflorum subsp. Gaudini (annual ryegrass, LOLMG), Phalaris minor Retz. (littleseed canarygrass, PHAMI), Poa annua L. (annual bluegrass, POAAN), Setaria pumila (Poir.) Roemer & J. A. Schultes (yellow foxtail, SETLU), Setaria viridis (L.) Beauv. (green foxtail, SETVI), Cirsium arvense (L.) Scop. (Canada thistle, CIRAR), Commelina benghalensis L. (tropical spiderwort, COMBE), Conyza canadensis (horseweed/marestail, ERICA), Conyza bonariensis (fleabane, ERIBO), Echinochloa crus-galli (L.) P. Beauv. (barnyardgrass, ECHCG), Euphorbia heterophylla L. (wild poinsettia, EPHHL), Conyza species (fleabane, ERISS), Conyza sumatrensis (Guernsey fleabane, ERISU), Ipomoea grandifolia (Corda-de-viola, IAQGR), Galium aparine L. (catchweed bedstraw, GALAP), Kochia scoparia (L.) Schrad. (kochia, KCHSC), Lamium purpureum L. (purple deadnettle, LAMPU), Matricaria recutita L. (wild chamomile, MATCH), Matricaria matricarioides (Less.) Porter (pineappleweed, MATMT), Papaver rhoeas L. (common poppy, PAPRH), Polygonum convolvulus L. (wild buckwheat, POLCO), Salsola tragus L. (Russian thistle, SASKR), Stellaria media (L.) Vill. (common chickweed, STEME), Veronica persica Poir. (Persian speedwell, VERPE), Viola arvensis Murr. (field violet, VIOAR), or Viola tricolor L. (wild violet, VIOTR).

In some embodiments, the compositions and methods provided herein are utilized to control undesirable vegetation in fallow fields and fallow beds between crops or before planting in rice. In certain embodiments, the undesirable vegetation is Brachiaria platyphylla (Groseb.) Nash (broadleaf signalgrass, BRAPP), Digitaria sanguinalis (L.) Scop. (large crabgrass, DIGSA), Echinochloa crus-galli (L.) P. Beauv. (barnyardgrass, ECHCG), Echinochloa colonum (L.) LINK (junglerice, ECHCO), Echinochloa oryzoides (Ard.) Fritsch (early watergrass, ECHOR), Echinochloa oryzicola (Vasinger) Vasinger (late watergrass, ECHPH), Conyza species (fleabane, ERISS), Ischaemum rugosum Salisb. (saramollagrass, ISCRU), Leptochloa chinensis (L.) Nees (Chinese sprangletop, LEFCH), Leptochloa fascicularis (Lam.) Gray (bearded sprangletop, LEFFA), Leptochloa panicoides (Presl.) Hitchc. (Amazon sprangletop, LEFPA), Panicum dichotomiflorum (L.) Michx. (Fall panicum, PANDI), Paspalum dilatatum Poir. (dallisgrass, PASDI), Cyperus difformis L. (smallflower flatsedge, CYPDI), Cyperus esculentus L. (yellow nutsedge, CYPES), Cyperus iria L. (rice flatsedge, CYPIR), Cyperus rotundus L. (purple nutsedge, CYPRO), Eleocharis species (ELOSS), Fimbristylis miliacea (L.) Vahl (globe fringerush, FIMMI), Schoenoplectus juncoides Roxb. (Japanese bulrush, SPCJU), Schoenoplectus maritimus L. (sea clubrush, SCPMA), Schoenoplectus mucronatus L. (ricefield bulrush, SCPMU), Aeschynomene species, (jointvetch, AESSS), Alternanthera philoxeroides (Mart.) Griseb. (alligatorweed, ALRPH), Alisma plantago-aquatica L. (common waterplantain, ALSPA), Amaranthus species, (pigweeds and amaranths, AMASS), Ammannia coccinea Rottb. (redstem, AMMCO), Eclipta alba (L.) Hassk. (American false daisy, ECLAL), Heteranthera limosa (SW.) Willd./Vahl (ducksalad, HETLI), Heteranthera reniformis R. & P. (roundleaf mudplantain, HETRE), Ipomoea hederacea (L.) Jacq. (ivyleaf morningglory, IPOHE), Lindernia dubia (L.) Pennell (low false pimpernel, LIDDU), Monochoria korsakowii Regel & Maack (monochoria, MOOKA), Monochoria vaginalis (Burm. F.) C. Presl ex Kuhth, (monochoria, MOOVA), Murdannia nudiflora (L.) Brenan (doveweed, MUDNU), Polygonum pensylvanicum L., (Pennsylvania smartweed, POLPY), Polygonum persicaria L. (ladysthumb, POLPE), Polygonum hydropiperoides Michx. (mild smartweed, POLHP), Rotala indica (Willd.) Koehne (Indian toothcup, ROTIN), Sagittaria species, (arrowhead, SAGSS), Sesbania exaltata (Raf.) Cory/Rydb. Ex Hill (hemp sesbania, SEBEX), or Sphenoclea zeylanica Gaertn. (gooseweed, SPDZE).

In some embodiments, the compositions and methods provided herein are utilized to control undesirable vegetation in fallow fields and fallow beds between crops or before planting range and pasture and forestry. In certain embodiments, the undesirable vegetation is Ambrosia artemisiifolia L. (common ragweed, AMBEL), Cassia obtusifolia (sickle pod, CASOB), Centaurea maculosa auct. non Lam (spotted knapweed, CENMA), Cirsium arvense (L.) Scop. (Canada thistle, CIRAR), Commelina benghalensis L. (tropical spiderwort, COMBE), Conyza canadensis (horseweed/marestail, ERICA), Conyza bonariensis (fleabane, ERIBO), Echinochloa crus-galli (L.) P. Beauv. (barnyardgrass, ECHCG), Euphorbia heterophylla L. (wild poinsettia, EPHHL), Conyza species (fleabane, ERISS), Conyza sumatrensis (Guernsey fleabane, ERISU), Ipomoea grandifolia (Corda-de-viola, IAQGR), Convolvulus arvensis L. (field bindweed, CONAR), Euphorbia esula L. (leafy spurge, EPHES), Lactuca serriola L./Torn. (prickly lettuce, LACSE), Plantago lanceolata L. (buckhorn plantain, PLALA), Rumex obtusifolius L. (broadleaf dock, RUMOB), Sida spinosa L. (prickly sida, SIDSP), Sinapis arvensis L. (wild mustard, SINAR), Sonchus arvensis L. (perennial sowthistle, SONAR), Solidago species (goldenrod, SOOSS), Taraxacum officinale G. H. Weber ex Wiggers (dandelion, TAROF), Trifolium repens L. (white clover, TRFRE), or Urtica dioica L. (common nettle, URTDI).

In some embodiments, the compositions and methods provided herein are utilized to control undesirable vegetation in fallow fields and fallow beds between crops, before planting crops, or within row crops and vegetable crops. In certain embodiments, the undesirable vegetation is Alopecurus myosuroides Huds. (blackgrass, ALOMY), Avena fatua L. (wild oat, AVEFA), Brachiaria platyphylla (Groseb.) Nash (broadleaf signalgrass, BRAPP), Commelina benghalensis L. (tropical spiderwort, COMBE), Conyza species (fleabane, ERISS), Conyza sumatrensis (Guernsey fleabane, ERISU), Ipomoea grandifolia (Corda-de-viola, IAQGR), Digitaria sanguinalis (L.) Scop. (large crabgrass, DIGSA), Echinochloa crus-galli (L.) P. Beauv. (barnyardgrass, ECHCG), Echinochloa colonum (L.) Link (junglerice, ECHCO), Lolium multiflorum Lam. (Italian ryegrass, LOLMU), Panicum dichotomiflorum Michx. (Fall panicum, PANDI), Panicum miliaceum L. (wild-proso millet, PANMI), Setaria faberi Herrm. (giant foxtail, SETFA), Setaria viridis (L.) Beauv. (green foxtail, SETVI), Sorghum halepense (L.) Pers. (Johnsongrass, SORHA), Sorghum bicolor (L.) Moench ssp. Arundinaceum (shattercane, SORVU), Cyperus esculentus L. (yellow nutsedge, CYPES), Cyperus rotundus L. (purple nutsedge, CYPRO), Abutilon theophrasti Medik. (velvetleaf, ABUTH), Amaranthus species (pigweeds and amaranths, AMASS), Ambrosia artemisiifolia L. (common ragweed, AMBEL), Ambrosia psilostachya DC. (Western ragweed, AMBPS), Ambrosia trifida L. (giant ragweed, AMBTR), Asclepias syriaca L. (common milkweed, ASCSY), Chenopodium album L. (common lambsquarters, CHEAL), Cirsium arvense (L.) Scop. (Canada thistle, CIRAR), Commelina benghalensis L. (tropical spiderwort, COMBE), Datura stramonium L. (jimsonweed, DATST), Daucus carota L. (wild carrot, DAUCA), Euphorbia heterophylla L. (wild poinsettia, EPHHL), Erigeron bonariensis L. (hairy fleabane, ERIBO), Erigeron canadensis L. (Canadian fleabane, ERICA), Helianthus annuus L. (common sunflower, HELAN), Jacquemontia tamnifolia (L.) Griseb. (smallflower morningglory, IAQTA), Ipomoea hederacea (L.) Jacq. (ivyleaf morningglory, IPOHE), Ipomoea lacunosa L. (white morningglory, IPOLA), Lactuca serriola L./Torn. (prickly lettuce, LACSE), Portulaca oleracea L. (common purslane, POROL), Sida spinosa L. (prickly sida, SIDSP), Sinapis arvensis L. (wild mustard, SINAR), Solanum ptychanthum Dunal (eastern black nightshade, SOLPT), or Xanthium strumarium L. (common cocklebur, XANST).

In some embodiments, the compositions and methods provided herein are utilized to control undesirable vegetation consisting of grass, broadleaf and sedge weeds.

In certain embodiments of the compositions and methods described herein, the compound of formula (I) or salt or ester thereof is used in combination with a cell membrane disruptor herbicide or an agriculturally acceptable salt or ester thereof. With regard to the compositions, in some embodiments, the weight ratio of the compound of formula (I) or salt or ester thereof to a cell membrane disruptor herbicide or an agriculturally acceptable salt or ester thereof is within the range from about 1:4800 to about 100:1. In certain embodiments, the weight ratio of the compound of formula (I) or salt or ester thereof to a cell membrane disruptor herbicide or an agriculturally acceptable salt or ester thereof is within the range from about 1:1150 to about 30:1. In certain embodiments, the compositions provided herein contain the methyl ester of the compound of formula (I). In one embodiment, the composition contains the methyl ester of the compound of formula (I), and a disruptor herbicide or an agriculturally acceptable salt or ester thereof wherein the weight ratio of the compound of formula (I) to a disruptor herbicide or an agriculturally acceptable salt or ester thereof is from about 1:400 to about 4:1.

With respect to the methods, in certain embodiments, the methods include contacting the undesirable vegetation or locus thereof or applying to the soil or water to prevent the emergence or growth of vegetation a composition described herein. In some embodiments, the composition is applied at an application rate from about 1 grams active ingredient per hectare (g ai/ha) to about 2450 g ai/ha based on the total amount of active ingredients in the composition. In certain embodiments, the composition is applied at an application rate from about 3 g ai/ha to about 2330 g ai/ha based on the total amount of active ingredients in the composition. In other embodiments, the composition is applied at an application rate from about 10 g ai/ha to about 2020 g ai/ha based on the total amount of active ingredients in the composition.

In some embodiments, the methods include contacting the undesirable vegetation or locus thereof or applying to the soil or water to prevent the emergence or growth of vegetation with a compound of formula (I) or salt or ester thereof and a cell membrane disruptor herbicide or an agriculturally acceptable salt or ester thereof e.g., sequentially or simultaneously. In some embodiments, the compound of formula (I) or salt or ester thereof is applied at a rate from about 0.5 grams acid equivalent per hectare (g ae/ha) to about 50 g ae/ha, and the cell membrane disruptor herbicide or an agriculturally acceptable salt or ester thereof is applied at a rate from about 0.5 g ai/ha to about 2400 g ai/ha. In some embodiments, the compound of formula (I) or salt or ester thereof is applied at a rate from about 2 g ae/ha to about 30 g ae/ha, and the cell membrane disruptor herbicide or an agriculturally acceptable salt or ester thereof is applied at a rate from about 1 g ai/ha to about 2300 g ai/ha. In certain embodiments, the methods utilize the compound of formula (I) or its methyl ester and a cell membrane disruptor herbicide or an agriculturally acceptable salt or ester thereof. In one embodiment, the methods utilize the methyl ester of the compound of formula (I) and a cell membrane disruptor herbicide or an agriculturally acceptable salt or ester thereof, wherein the methyl ester of the compound of formula (I) is applied at a rate from about 5 g ae/ha to about 20 g ae/ha, and a cell membrane disruptor herbicide or an agriculturally acceptable salt or ester thereof is applied at a rate from about 5 g ai/ha to about 2000 g ai/ha.

In certain embodiments of the compositions and methods described herein, the compound of formula (I) or salt or ester thereof is used in combination with a photosystem I (PS I) inhibitor herbicide or an agriculturally acceptable salt thereof. With regard to the compositions, in some embodiments, the weight ratio of the compound of formula (I) or salt or ester thereof to a photosystem I (PS I) inhibitor herbicide or an agriculturally acceptable salt thereof is within the range from about 1:4480 to about 2.5:1. In certain embodiments, the compositions provided herein contain the methyl ester of the compound of formula (I) and a PS I inhibitor herbicide or an agriculturally acceptable salt thereof. In one embodiment, the composition comprises the methyl ester of the compound of formula (I) and a PS I inhibitor herbicide or an agriculturally acceptable salt thereof wherein the weight ratio of the compound of formula (I) to a PS I inhibitor herbicide or an agriculturally acceptable salt thereof is from about 1:900 to about 0.5:1. In one embodiment, the composition contains the methyl ester of the compound of formula (I), a PS I inhibitor herbicide or an agriculturally acceptable salt thereof, wherein the weight ratio of the methyl ester of the compound of formula (I) to a PS I inhibitor herbicide or an agriculturally acceptable salt thereof is from about 1:180 to about 0.2:1.

With respect to the methods, in certain embodiments, the methods include contacting the undesirable vegetation or locus thereof or applying to the soil or water to prevent the emergence or growth of vegetation a composition described herein. In some embodiments, the composition is applied at an application rate from about 20 grams active ingredient per hectare (g ai/ha) to about 2290 g ai/ha based on the total amount of active ingredients in the composition. In certain embodiments, the composition is applied at an application rate from about 62 g ai/ha to about 1830 g ai/ha based on the total amount of active ingredients in the composition. In other embodiments, the composition is applied at an application rate from about 105 g ai/ha to about 920 g ai/ha based on the total amount of active ingredients in the composition.

In some embodiments, the methods include contacting the undesirable vegetation or locus thereof or applying to the soil or water to prevent the emergence or growth of vegetation with a compound of formula (I) or salt or ester thereof and a PS I inhibitor herbicide or an agriculturally acceptable salt thereof e.g., sequentially or simultaneously. In some embodiments, the compound of formula (I) or salt or ester thereof is applied at a rate from about 0.5 grams acid equivalent per hectare (g ae/ha) to about 50 g ae/ha, and the PS I inhibitor herbicide or an agriculturally acceptable salt thereof is applied at a rate from about 20 g ai/ha to 2240 g ai/ha. In some embodiments, the compound of formula (I) or salt or ester thereof is applied at a rate from about 2 g ae/ha to about 30 g ae/ha, and the PS I inhibitor herbicide or an agriculturally acceptable salt thereof is applied at a rate from about 60 g ai/ha to 1800 g ai/ha. In one embodiment, the methods utilize the methyl ester of the compound of formula (I) and a PS I inhibitor herbicide or an agriculturally acceptable salt thereof, wherein the methyl ester of the compound of formula (I) is applied at a rate from about 5 g ae/ha to about 20 g ae/ha, and the PS I inhibitor herbicide or an agriculturally acceptable salt thereof is applied at a rate from about 100 g ai/ha to about 900 g ai/ha.

In certain embodiments of the compositions and methods described herein, the compound of formula (I) or salt or ester thereof is used in combination with paraquat or an agriculturally acceptable salt thereof. With regard to the compositions, in some embodiments, the weight ratio of the compound of formula (I) or salt or ester thereof to paraquat or an agriculturally acceptable salt thereof is within the range from about 1:4480 to about 2.5:1. In certain embodiments, the compositions provided herein contain the methyl ester of the compound of formula (I) and paraquat or an agriculturally acceptable salt thereof. In one embodiment, the composition comprises the methyl ester of the compound of formula (I) and paraquat or an agriculturally acceptable salt thereof wherein the weight ratio of the compound of formula (I) to paraquat or an agriculturally acceptable salt thereof is from about 1:900 to about 0.5:1. In one embodiment, the composition contains the methyl ester of the compound of formula (I) and paraquat or an agriculturally acceptable salt thereof wherein the weight ratio of the methyl ester of the compound of formula (I) to paraquat or an agriculturally acceptable salt thereof is from about 1:80 to about 0.2:1.

With respect to the methods, in certain embodiments, the methods include contacting the undesirable vegetation or locus thereof or applying to the soil or water to prevent the emergence or growth of vegetation a composition described herein. In some embodiments, the composition is applied at an application rate from about 20.5 grams active ingredient per hectare (g ai/ha) to about 2290 g ai/ha based on the total amount of active ingredients in the composition. In certain embodiments, the composition is applied at an application rate from about 62 g ai/ha to about 1830 g ai/ha based on the total amount of active ingredients in the composition. In other embodiments, the composition is applied at an application rate from about 105 g ai/ha to about 420 g ai/ha based on the total amount of active ingredients in the composition.

In some embodiments, the methods include contacting the undesirable vegetation or locus thereof or applying to the soil or water to prevent the emergence or growth of vegetation with a compound of formula (I) or salt or ester thereof and paraquat or an agriculturally acceptable salt thereof e.g., sequentially or simultaneously. In some embodiments, the compound of formula (I) or salt or ester thereof is applied at a rate from about 0.5 grams acid equivalent per hectare (g ae/ha) to about 50 g ae/ha, and the paraquat is applied at a rate from about 20 g ai/ha to 2240 g ai/ha. In some embodiments, the compound of formula (I) or salt or ester thereof is applied at a rate from about 2 g ae/ha to about 30 g ae/ha, and the paraquat or an agriculturally acceptable salt thereof is applied at a rate from about 60 g ai/ha to 1800 g ai/ha. In one embodiment, the methods utilize the methyl ester of the compound of formula (I) and paraquat or an agriculturally acceptable salt thereof, wherein the methyl ester of the compound of formula (I) is applied at a rate from about 5 g ae/ha to about 20 g ae/ha and the paraquat or an agriculturally acceptable salt thereof is applied at a rate from about 100 g ai/ha to about 400 g ai/ha.

In certain embodiments of the compositions and methods described herein, the compound of formula (I) or salt or ester thereof is used in combination with a a protoporphyrinogen oxidase (PPO) inhibitor herbicide or an agriculturally acceptable salt or ester thereof. With regard to the compositions, in some embodiments, the weight ratio of the compound of formula (I) or salt or ester thereof to a PPO inhibitor herbicide or an agriculturally acceptable salt or ester thereof is within the range from about 1:4800 to about 100:1. In certain embodiments, the compositions provided herein contain the methyl ester of the compound of formula (I) and a PPO inhibitor herbicide or an agriculturally acceptable salt or ester thereof. In one embodiment, the composition comprises the methyl ester of the compound of formula (I) and a PPO inhibitor herbicide or an agriculturally acceptable salt or ester thereof wherein the weight ratio of the compound of formula (I) to a PPO inhibitor herbicide or an agriculturally acceptable salt or ester thereof is from about 1:1150 to about 30:1. In one embodiment, the composition contains the methyl ester of the compound of formula (I) and a PPO inhibitor herbicide or an agriculturally acceptable salt or ester thereof wherein the weight ratio of the methyl ester of the compound of formula (I) to a PPO inhibitor herbicide or an agriculturally acceptable salt or ester thereof is from about 1:400 to about 4:1.

With respect to the methods, in certain embodiments, the methods include contacting the undesirable vegetation or locus thereof or applying to the soil or water to prevent the emergence or growth of vegetation a composition described herein. In some embodiments, the composition is applied at an application rate from about 1 grams active ingredient per hectare (g ai/ha) to about 2450 g ai/ha based on the total amount of active ingredients in the composition. In certain embodiments, the composition is applied at an application rate from about 3 g ai/ha to about 2330 g ai/ha based on the total amount of active ingredients in the composition. In other embodiments, the composition is applied at an application rate from about 10 g ai/ha to about 2020 g ai/ha based on the total amount of active ingredients in the composition.

In some embodiments, the methods include contacting the undesirable vegetation or locus thereof or applying to the soil or water to prevent the emergence or growth of vegetation with a compound of formula (I) or salt or ester thereof and a PPO inhibitor herbicide or an agriculturally acceptable salt or ester thereof e.g., sequentially or simultaneously. In some embodiments, the compound of formula (I) or salt or ester thereof is applied at a rate from about 0.5 grams acid equivalent per hectare (g ae/ha) to about 50 g ae/ha and the PPO inhibitor herbicide or an agriculturally acceptable salt or ester thereof is applied at a rate from about 0.5 g ai/ha to 2400 g ai/ha. In some embodiments, the compound of formula (I) or salt or ester thereof is applied at a rate from about 2 g ae/ha to about 30 g ae/ha and the a PPO inhibitor herbicide or an agriculturally acceptable salt or ester thereof is applied at a rate from about 1 g ai/ha to 2300 gr ai/ha. In one embodiment, the methods utilize the methyl ester of the compound of formula (I) and a PPO inhibitor herbicide or an agriculturally acceptable salt or ester thereof, wherein the methyl ester of the compound of formula (I) is applied at a rate from about 5 g ae/ha to about 20 g ae/ha and the a PPO inhibitor herbicide or an agriculturally acceptable salt or ester thereof is applied at a rate from about 5 g ai/ha to about 2000 g ai/ha.

In certain embodiments of the compositions and methods described herein, the compound of formula (I) or salt or ester thereof is used in combination with saflufenacil. With regard to the compositions, in some embodiments, the weight ratio of the compound of formula (I) or salt or ester thereof to saflufenacil is within the range from about 1:400 to about 100:1. In one embodiment, the composition comprises the methyl ester of the compound of formula (I) and saflufenacil wherein the weight ratio of the compound of formula (I) to saflufenacil is from about 1:75 to about 30:1. In one embodiment, the composition contains the methyl ester of the compound of formula (I) and saflufenacil wherein the weight ratio of the methyl ester of the compound of formula (I) to saflufenacil is from about 1:9.8 to about 2.3:1.

With respect to the methods, in certain embodiments, the methods include contacting the undesirable vegetation or locus thereof or applying to the soil or water to prevent the emergence or growth of vegetation a composition described herein. In some embodiments, the composition is applied at an application rate from about 1 grams active ingredient per hectare (g ai/ha) to about 250 g ai/ha based on the total amount of active ingredients in the composition. In certain embodiments, the composition is applied at an application rate from about 3 g ai/ha to about 180 g ai/ha based on the total amount of active ingredients in the composition. In other embodiments, the composition is applied at an application rate from about 13 g ai/ha to about 69 g ai/ha based on the total amount of active ingredients in the composition.

In some embodiments, the methods include contacting the undesirable vegetation or locus thereof or applying to the soil or water to prevent the emergence or growth of vegetation with a compound of formula (I) or salt or ester thereof and saflufenacil e.g., sequentially or simultaneously. In some embodiments, the compound of formula (I) or salt or ester thereof is applied at a rate from about 0.5 grams acid equivalent per hectare (g ae/ha) to about 50 g ae/ha and the saflufenacil is applied at a rate from about 0.5 g ai/ha to 200 g ai/ha. In some embodiments, the compound of formula (I) or salt or ester thereof is applied at a rate from about 2 g ae/ha to about 30 g ae/ha, and the saflufenacil is applied at a rate from about 1 g ai/ha to 150 g ai/ha. In one embodiment, the methods utilize the methyl ester of the compound of formula (I) and saflufenacil, wherein the methyl ester of the compound of formula (I) is applied at a rate from about 5 g ae/ha to about 20 g ae/ha and the saflufenacil is applied at a rate from about 8.75 g ai/ha to about 49 g ai/ha.

The components of the mixtures described herein can be applied either separately or as part of a multipart herbicidal system. In some embodiments of the methods described herein, the active ingredients are applied simultaneously, including, e.g., in the form of a composition. In some embodiments, the active ingredients are applied sequentially, e.g., within 5, 10, 15, or 30 minutes of each other; or 1, 2, 3, 4, 5, 10, 12, 24, 48, 72 hour(s) of each other; or 1 week of each other.

The present compositions can be applied to weeds and other undesirable vegetation, or to their locus thereof by the use of conventional ground or aerial dusters, sprayers, and granule applicators and by other conventional means known to those skilled in the art.

The described embodiments and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

### EXAMPLES

### Example 1. Evaluation of Postemergence Herbicidal Activity

Treatments consisted of the methyl ester of 4-amino-3-chloro-6-(4-chloro-2-fluoro-3-methoxyphenyl)pyridine-2-carboxylic acid (Cmpd 1) tankmixed with saflufenacil or paraquat. The form of compound of formula (I) was applied on an acid equivalent basis, and saflufenacil and paraquat were applied on an active ingredient basis. Saflufenacil was applied as a commercial formulation containing 700 grams per kilogram (g/kg). Paraquat was applied as a commercial formulation containing 200 g/L.

Field trials were conducted under standard field conditions. Plot sizes varied from 2 meters (m) wide by 5 m long. Treatments were applied with small plot backpack sprayers, using pressurized carbon dioxide (CO2) as propellant. Pressure varied from 40-45 pounds per square inch (PSI), with spray tips typically of a Flat Fan type applying water diluent at 100 liters per hectare (L/ha). Boom size was 2 m in width. Weed sizes varied, but all treatments were applied as post-emergence treatments to weeds from 2 leaf to 2 tiller growth stages varying from 2-15 inches in height. No additional adjuvant was mixed with the treatments applied in the field.

Results in Tables 1 to 2 are field trial results for foliar applied compositions. The values reported are percent (%) control visually rated. The predicted percent (%) weed contol was determined using Colby's equation.

The following abbreviations are used in Tables 1 to 2:

ERISS=Conyza species (fleabane, ERISS)
g ae/ha=grams acid equivalent per hectare
g ai/ha=grams active ingredient per hectare
Mean % weed control=observed value of percent (%) control rated visually
Predicted mean % weed control=expected value of percent (%) control as calculated by Colby's equation
Cmpd 1=methyl 4-amino-3-chloro-6-(4-chloro-2-fluoro-3-methoxyphenyl)pyridine-2-carboxylate, halauxifen-methyl, or ARYLEX®
DAA=days after application

TABLE 1
Weed control from post-emergent application of Cmpd 1 and saflufenacil

| | | | | | | Combination | |
|---|---|---|---|---|---|---|---|
| | | Cmpd 1 | | Saflufenacil | | Measured | Predicted |
| Weed | | | Mean % | | Mean % | mean % | mean % |
| Bayer | Evaluation | | weed | | weed | weed | weed |
| Code | Interval | g ae/ha | control | g ai/ha | control | control | control |
| ERISS | 17DAA | 5 | 33.75 | 49 | 52.5 | 82.5 | 68.94 |
| ERISS | 28DAA | 5 | 33.75 | 49 | 66.25 | 92.5 | 77.62 |
| ERISS | 43DAA | 5 | 40 | 49 | 77.5 | 98.75 | 86.38 |
| ERISS | 17DAA | 5 | 33.75 | 49 | 66.25 | 92.5 | 77.62 |

TABLE 2
Weed control from post-emergent application of Cmpd 1 and paraquat

| | | | | | | Combination | |
|---|---|---|---|---|---|---|---|
| | | Cmpd 1 | | Paraquat | | Measured | Predicted |
| Weed | | | Mean % | | Mean % | mean % | mean % |
| Bayer | Evaluation | | weed | | weed | weed | weed |
| Code | Interval | g ae/ha | control | g ai/ha | control | control | control |
| ERISS | 17DAA | 5 | 33.75 | 400 | 32.5 | 71.25 | 55.44 |
| ERISS | 28DAA | 5 | 33.75 | 400 | 52.5 | 75 | 68.56 |
| ERISS | 43DAA | 5 | 40 | 400 | 60 | 82.5 | 75.94 |
| ERISS | 17DAA | 5 | 33.75 | 400 | 52.5 | 81.25 | 68.56 |
| ERISS | 28DAA | 5 | 46.25 | 400 | 55 | 98.25 | 75.75 |
| ERISS | 43DAA | 5 | 57.5 | 400 | 65 | 97.5 | 85.12 |

What is claimed is:

1. A herbicidal tank mix composition comprising a herbicidally effective amount of (a) a compound of the formula (I) 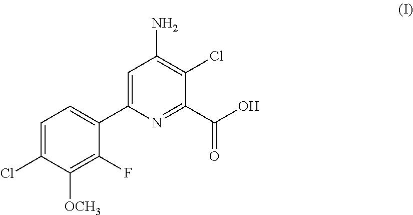 [SMILES: COc1c(Cl)ccc(-c2cc(N)c(Cl)c(C(=O)O)n2)c1F]
or an agriculturally acceptable salt or ester thereof and (b) a cell membrane disruptor herbicide or an agriculturally acceptable salt or ester thereof, wherein (b) is not carfentrazone or an agriculturally acceptable salt or ester thereof.

2. The composition of claim 1, wherein no additional herbicidal active ingredients are present in the composition.

3. The composition of claim 1, wherein (a) is a C₁-C₄ alkyl or benzyl ester of compound (I).

4. The composition of claim 3, wherein (a) is a methyl ester of compound (I).

5. The composition of claim 1, wherein (a) is the carboxylic acid of formula (I).

6. The composition of claim 1, wherein (a) is the methyl ester of the compound of formula (I) and (b) is a photosystem I (PS I) inhibitor or an agriculturally acceptable salt thereof.

7. The composition of claim 6, wherein the PS I inhibitor is selected from the group consisting of diquat, paraquat, agriculturally acceptable salts thereof, and combinations thereof.

8. The composition of claim 1, wherein (a) is the methyl ester of the compound of formula (I) and (b) is a protoporphyrinogen oxidase (PPO) inhibitor or an agriculturally acceptable salt or ester thereof.

9. The composition of claim 8, wherein the PPO inhibitor is selected from the group consisting of acifluorfen, bifenox, chlomethoxyfen, fluoroglycofen, fomesafen, halosafen, lactofen, oxyfluorfen, fluazolate, pyraflufen, cinidon, flumioxazin, flumiclorac, fluthiacet, thidiazimin, oxadiazon, oxadiargyl, azafenidin, sulfentrazone, pentoxazone, trifludimoxazin, benzfendizone, butafenacil, saflufenacil, pyraclonil, profluazol, flufenpyr, agriculturally acceptable salts or esters thereof, and combinations thereof.

10. The composition of claim 1, wherein (b) is a photosystem I (PS I) inhibitor or an agriculturally acceptable salt thereof and the weight ratio of (a) to (b) is from about 1:4480 to about 2.5:1.

11. The composition of claim 10, wherein (b) is a photosystem I (PS I) inhibitor herbicide or an agriculturally acceptable salt thereof and the weight ratio of (a) to (b) is from about 1:900 to about 0.5:1.

12. The composition of claim 10, wherein (b) is a photosystem I (PS I) inhibitor herbicide or an agriculturally acceptable salt thereof and the weight ratio of (a) to (b) is from about 1:180 to about 0.2:1.

13. The composition of claim 1, wherein (a) is the methyl ester of the compound of formula (I) and (b) is paraquat or an agriculturally acceptable salt thereof and the weight ratio of (a) to (b) is from about 1:4480 to about 2.5:1.

14. The composition of claim 13, wherein (a) is the methyl ester of the compound of formula (I) and (b) is paraquat or an agriculturally acceptable salt thereof and the weight ratio of (a) to (b) is from about 1:900 to about 0.5:1.

15. The composition of claim 13, wherein (a) is the methyl ester of the compound of formula (I) and (b) is paraquat or an agriculturally acceptable salt thereof and the weight ratio of (a) to (b) is from about 1:80 to about 0.2:1.

16. The composition of claim 1, wherein (b) is a protoporphyrinogen oxidase (PPO) inhibitor or an agriculturally acceptable salt thereof and the weight ratio of (a) to (b) is from about 1:4800 to about 100:1.

17. The composition of claim 16, wherein (b) is a photosystem I (PS I) inhibitor herbicide or an agriculturally acceptable salt thereof and the weight ratio of (a) to (b) is from about 1:1150 to about 30:1.

18. The composition of claim 16, wherein (b) is a photosystem I (PS I) inhibitor herbicide or an agriculturally acceptable salt thereof and the weight ratio of (a) to (b) is from about 1:400 to about 4:1.

19. The composition of claim 1, wherein (a) is the methyl ester of the compound of formula (I) and (b) is saflufenacil and the weight ratio of the compound of (a) to (b) is from about 1:400 to about 100:1.

20. The composition of claim 19, wherein (a) is the methyl ester of the compound of formula (I) and (b) is saflufenacil and the weight ratio of (a) to (b) is from about 1:75 to about 30:1.

21. The composition of claim 19, wherein (a) is the methyl ester of the compound of formula (I) and (b) is saflufenacil and the weight ratio of (a) to (b) is from about 1:9.8 to about 2.3:1.

22. The composition of claim 1, further comprising a herbicide safener.

23. The composition of claim 1, further comprising an agriculturally acceptable adjuvant or carrier.

24. A method of controlling undesirable vegetation which comprises applying a herbicidally effective amount of a herbicidal composition comprising:
(a) a compound of the formula (I) 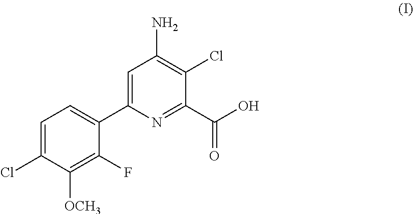 [SMILES: COc1c(Cl)ccc(-c2cc(N)c(Cl)c(C(=O)O)n2)c1F]
or an agriculturally acceptable salt or ester thereof and (b) a cell membrane disruptor herbicide or an agriculturally acceptable salt or ester thereof, wherein (b) is not carfentrazone or an agriculturally acceptable salt or ester thereof.

25. The method of claim 24, wherein no additional herbicidal active ingredients are present in the composition.

26. The method of claim 24 wherein the undesirable vegetation is controlled in soybean, cotton, sunflower, winter/spring oilseed rape, winter/spring canola, vegetables, ornamentals, rice, wheat, teff, triticale, barley, oats, rye, sorghum, corn/maize, sunflower, row crops, pastures, grasslands, rangelands, fallowland, sugarcane, turf, tree and vine orchards, aquatics, forestry, industrial vegetation management (IVM) and rights-of-way; burndown and control of weeds in fallow fields and fallow beds between the planting of crops, such as soybean, corn, cotton, sunflower, cereals, rice, winter/spring oilseed rape, winter/spring canola, corn/maize and sorghum.

27. The method of claim 24, wherein the composition is applied for burndown and control of undesirable vegetation in fallow fields and fallow-beds.

28. The method of claim 24, wherein the composition is used to control weeds just prior to or after planting soybeans.

29. The method of claim 24, wherein the undesirable vegetation is immature.

30. The method of claim 24, wherein the (a) and (b) are applied pre-emergence to the weed or crop.

31. The method of claim 24, wherein the (a) and (b) are applied post-emergence to the weed or crop.

32. The method of claim 24, wherein the undesirable vegetation is controlled in a crop that is tolerant to glyphosate, glufosinate, dicamba, phenoxy auxins, pyridyloxy auxins, aryloxyphenoxypropionates, acetyl CoA carboxylase (ACCase) inhibitors, imidazolinones, acetolactate synthase (ALS) inhibitors, 4-hydroxyphenyl-pyruvate dioxygenase (HPPD) inhibitors, protoporphyrinogen oxidase (PPO) inhibitors, triazines, or bromoxynil.

33. The method of claim 32, wherein the tolerant crop possesses single or multiple or stacked traits conferring tolerance to single or multiple herbicides or single or multiple modes of action.

34. The method of claim 24, wherein the undesirable vegetation comprises a herbicide resistant or tolerant weed.

35. The method of claim 34, wherein the resistant or tolerant weed is a biotype with resistance or tolerance to single or multiple herbicides, single or multiple chemical classes, or single or multiple herbicide modes of action.

36. The method of claim 35, wherein the resistant or tolerant weed is a biotype resistant or tolerant to acetolactate synthase (ALS) inhibitors, photosystem II inhibitors, acetyl CoA carboxylase (ACCase) inhibitors, synthetic auxins, photosystem I inhibitors, 5-enolpyruvylshikimate-3-phosphate (EPSP) synthase inhibitors, microtubule assembly inhibitors, lipid synthesis inhibitors, protoporphyrinogen oxidase (PPO) inhibitors, carotenoid biosynthesis inhibitors, very long chain fatty acid (VLCFA) inhibitors, phytoene desaturase (PDS) inhibitors, glutamine synthetase inhibitors, 4-hydroxyphenyl-pyruvate-dioxygenase (HPPD) inhibitors, mitosis inhibitors, cellulose biosynthesis inhibitors, herbicides with multiple modes of action, quinclorac, arylaminopropionic acids, difenzoquat, endothall, or organoarsenicals.

37. The method of claim 24, wherein the undesirable vegetation is Conyza species (fleabane, ERISS).

38. A herbicidal system comprising a first container comprising a concentrate comprising a compound of formula (I) 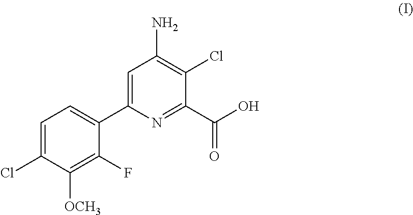 [SMILES: COc1c(Cl)ccc(-c2cc(N)c(Cl)c(C(=O)O)n2)c1F]
or an agriculturally acceptable salt or ester thereof, and a second container comprising a concentrate of a cell membrane disruptor herbicide or an agriculturally acceptable salt or ester thereof.

39. The system of claim 38, wherein the first and/or second container contains one or more adjuvants and/or carriers.
